(19) United States
(12) Patent Application Publication
(10) Pub. No.: US 2022/0259552 A1
(43) Pub. Date: Aug. 18, 2022

(54) METHOD FOR CONTROLLING A FERMENTATION PROCESS

(71) Applicant: Unibio A/S, Roskilde (DK)

(72) Inventors: Subir Kumar Nandy, Kongens Lyngby (DK); Leander Petersen, Troy, NY (US)

(21) Appl. No.: 17/618,232

(22) PCT Filed: Jun. 11, 2020
(86) PCT No.: PCT/EP2020/066198

§ 371 Date: Dec. 10, 2021

(30) Foreign Application Priority Data
Jun. 13, 2019 (DK) PA 2019 00714

(51) Int. Cl. C12N 1/30; C12M 1/00; C12M 1/34

(52) U.S. Cl. CPC C12N 1/30; C12M 41/32; C12M 29/18; C12M 29/06

(57) ABSTRACT

The present invention relates to a fermentation process for the fermentation of at least one microorganism, wherein the fermentation process comprises the steps of (a) allowing a fermentation broth comprising the at least one microorganism to flow in the fermentation reactor; (b) supplying a carbon-substrate to the fermentation reactor allowing the gaseous carbon-substrate to be dissolved, or partly dissolved, in the fermentation broth; (c) supplying a nitrogen-substrate to the fermentation reactor allowing the gaseous nitrogen-substrate to be dissolved, or partly dissolved, in the fermentation broth; and (d) maintaining a nitrate concentration of the fermentation broth below 0.035 g/l, and/or maintaining a nitrate concentration of the fermentation broth below 0.01 g nitrate/g biomass; wherein the at least one methanotrophic organism comprises at least one methanotrophic microorganism.

### TECHNICAL FIELD OF THE INVENTION

The present invention relates to a fermentation process and a fermentation reactor for improving biomass production. In particular, the present invention relates to a process and a fermentation reactor for fermenting methanotrophic organisms where the concentration of nitrate is strictly controlled in order to optimize the fermentation process.

### BACKGROUND OF THE INVENTION

A nitrogen-source is together with a carbon-source essential for microbial growth during fermentation. The nitrogen-source is required for microorganisms to synthesize proteins, nucleic acids, and other cellular components.

Depending on the enzyme capabilities of the microorganism, nitrogen may be provided as bulk protein, such as soy meal; as pre-digested polypeptides, such as peptone or tryptone; or as ammonia or nitrate salts. The choice of the nitrogen source may be important and depending on the product produced, since the cost of the nitrogen-source is an important factor.

Even the nitrogen-source is an essential component for the growth of microorganisms, it is also known in the art that methanotrophic microorganisms are highly sensitive to the load of nitrogen which may be influenced by the form of the nitrogen source, and the amount of the nitrogen source.

It is speculated in the prior art that this difference in tolerance of ammonia and nitrite may be due to different affinities of methane monooxygenase enzymes for e.g. ammonia or a toxic effect of nitrite.

Methane monooxygenase enzymes are responsible for rendering methanotrophy in methanotrophic microorganisms, and at the same time they carry out oxidations on available nitrogen-sources, leading to numerous co-metabolic by-products.

When growing methanotrophic microorganisms, like M. capsulatus, nitrogen-sources, such as ammonia, is readily oxidized by the methane monooxygenases of Methylococcus capsulatus even at low extracellular concentrations if methane is not in large excess.

To make a cost competitive single cell protein (SCP) product from Methylococcus capsulatus fermentation, ammonia is often used as the nitrogen-source for the fermentation. The solubility of ammonia in the aqueous fermentation broth is many orders of magnitude larger than the solubility of methane, which may be used as the carbon-source, making ammonia oxidation a real problem, even if the obvious immediate issue of gas to liquid mass transfer is addressed by the use of appropriate reactor design.

Hence, an improved fermentation process and/or fermentation reactor would be advantageous, and in particular, a more efficient and/or controlled fermentation process and/or fermentation reactor would be advantageous where the nitrogen-source is regulated in order to improve the production of methanotrophic biomass.

### SUMMARY OF THE INVENTION

Thus, an object of the present invention relates to an improved fermentation process for fermenting methanotrophic microorganisms, like Methylococcus capsulatus.

In particular, it is an object of the present invention to provide a more efficient and/or controlled fermentation process and/or fermentation reactor where the nitrogen-source may be regulated in order to improve the production of methanotrophic biomass, and a fermentation process and/or a fermentation reactor that solves the above mentioned problems of the prior art with controlling the level of nitrogen-source supplied during the fermentation to provide a nutrient for growth of the microorganisms, such as methanotrophic microorganisms, but at the same time avoid levels creating a competitive inhibitor of methane consumption.

Thus, one aspect of the invention relates to a fermentation process for fermenting a fermentation broth comprising at least one microorganism in a fermentation reactor, wherein the fermentation process comprises the steps of:
a) supplying a carbon-substrate to the fermentation reactor allowing the carbon-substrate to be dissolved, or partly dissolved, in the fermentation broth;
b) supplying a nitrogen-substrate to the fermentation reactor allowing the nitrogen-substrate to be dissolved, or partly dissolved, in the fermentation broth; and
c) maintaining a nitrate concentration of the fermentation broth below 0.035 g/l, and/or maintaining a nitrate concentration of the fermentation broth below 0.01 g nitrate/g biomass;
   wherein the at least one microorganism comprises at least one methanotrophic microorganism.

Another aspect of the present invention relates to a fermentation reactor comprising a loop-part and a top tank, said loop-part comprising a downflow part, connected to an upflow part via a U-part, wherein the top tank comprises:
(i) a first outlet connecting the top tank to the downflow part of the loop-part and allowing a fermentation liquid present in the top tank to flow from the top tank into the loop-part;
(ii) a first inlet connecting the top tank to the up-flow part of the loop-part, allowing fermentation liquid present in the loop-part to flow from the loop part into the top tank;
(iii) a vent tube for discharging effluent gasses from the top tank; and
(iv) a visual inspection means.
   wherein the fermentation reactor further comprises:
(v) at least one inlet for supplying a substrate comprising an ammonium compound; and
(vi) at least sensor for determining the concentration of nitrate in the fermentation broth;

### BRIEF DESCRIPTION OF THE FIGURES

FIG. 1 shows that the biomass production in the pilot plant (solid line) is decreasing over time as the nitrate production (dashed line) is increasing over time, and vice versa. This trend has been found in both laboratory tests, in a pilot plant as well as in a production plant.

The present invention will now be described in more detail in the following.

### DETAILED DESCRIPTION OF THE INVENTION

Accordingly, the inventors of the present invention found that since the nitrogen-source provided to a fermentation process may act both as a nutrient for growth of the microorganisms, such as the methanotrophic microorganisms, as well as a competitive inhibitor of methane consumption, e.g. by inhibiting the methane monooxygenase enzymes the concentration of nitrogen-source should be regulated and/or controlled in order to optimize the biomass production of methanotrophic microorganisms, such as methylococcus capsulatus.

Methylococcus capsulatus oxidizes ammonia (NH₃) or ammonium (NH₄⁺) to nitrite (NO₂⁻) where necessary enzymes involved are Methanemonooxygenase (MMO) which is capable of oxidising ammonia as well as methane and hydroxylamine oxidoreductase (HAO) by the following reactions. This reaction requires oxygen.

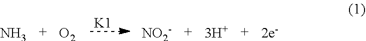

Without being bound by theory, the inventors of the present invention trust that nitrite produced by the methanotrophic microorganism, such as methylococcus capsulatus, in the first step (K1) of autotrophic nitrification is oxidized to nitrate by nitrite oxidoreductase (NXR) following the following reaction:

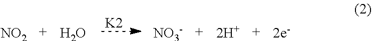

The rate of the above reactions (K1 and K2) and the reversible reactions is believed in combination to form nitrate substantially directly from ammonia with a very little trace of nitrite formation using methanotrophic microorganisms, such as M. capsulatus.

From experiments the inventors of the present invention surprisingly found that feeding nitrogen source, such as ammonia, to the fermentation of methanotrophic microorganisms, such as M. capsulatus, should be controlled and regulated in order to keep the nitrate concentration below a certain level in order to avoid a reduction in biomass development and/or provide a high biomass development.

In the present context the term “high biomass development” relates to a biomass concentration above 1 g/l; such as above 5 g/l; e.g. above 10 g/l; such as above 15 g/l; e.g. above 20 g/l; such as above 25 g/l; e.g. above 30 g/l; such as above 50 g/l; e.g. above 70 g/l; such as in the range of 1-100 g/l; e.g. in the range of 5-90 g/l; such as in the range of 10-80 g/l; e.g. in the range of 20-70 g/l; such as in the range of 30-65 g/l; e.g. in the range of 40-60 g/l; such as in the range of 45-55 g/l.

Therefore, nitrate formed by methanotrophic microorganisms, such as M. capsulatus, in the cultivation, e.g. using ammonia as a nitrogen-source may be used as a liable indicator of stress of the fermentation and therefore, the fermentation process can be controlled due to the operation by regulating the concentration of nitrate, e.g. by reducing the flow of nitrogen-source, in the fermentation reactor, or even stop the flow to zero L/min.

The inventors found that this way to control or regulate a fermentation process may be essential to ensure high productivity of methanotrophic biomass, such as M. capsulatus biomass, irrespective of running the fermentation process in batch, fed-batch or continuous mode.

This effect and importance of control and/or regulation have been demonstrated in the below experiment in both laboratory tests, in pilot scale as well as in a production/industrial.

Accordingly, the inventors of the present invention surprisingly found a fermentation process and a fermentation reactor where the nitrogen-source may be controlled and/or regulated in order to improve the production of methanotrophic biomass.

In a preferred embodiment of the present invention relates to a fermentation process for fermenting a fermentation broth comprising at least one microorganism in a fermentation reactor, wherein the fermentation process comprises the steps of:
d) supplying a carbon-substrate to the fermentation reactor allowing the carbon-substrate to be dissolved, or partly dissolved, in the fermentation broth;
e) supplying a nitrogen-substrate to the fermentation reactor allowing the nitrogen-substrate to be dissolved, or partly dissolved, in the fermentation broth; and
f) maintaining a nitrate concentration of the fermentation broth below 0.035 g/l, and/or maintaining a nitrate concentration of the fermentation broth below 0.01 g nitrate/g biomass;
   wherein the at least one microorganism comprises at least one methanotrophic microorganism.

The nitrate concentration of the fermentation broth during fermentation may be maintained below 0.035 g/l; such as below 0.033 g/l; e.g. below 0.03 g/l; such as below 0.028 g/l; e.g. below 0.025 g/l; such as below 0.022 g/l; e.g. below 0.02 g/l; such as below 0.018 g/l; e.g. below 0.015 g/l; such as below 0.01 g/l; e.g. below 0.005 g/l; such as below 0.01 g/l; e.g. at 0 g/l.

In an embodiment of the present invention the nitrate concentration of the fermentation broth during fermentation is in the range of 0-0.035 g/l; e.g. in the range of 0.001-0.033 g/l; such as in the range of 0.002-0.03 g/l; e.g. in the range of 0.003-0.025 g/l; such as in the range of 0.004-0.02 g/l; e.g. in the range of 0.005-0.015 g/l; such as in the range of 0.007-0.01 g/l.

The nitrogen-source may be a gaseous nitrogen-substrate or an aqueous nitrogen-substrate.

Preferably, the nitrogen-source may be selected from ammonia; ammonium compounds; and/or molecular nitrogen. Even more preferably, the nitrogen-source is ammonia.

The ammonium compound may be selected from ammonium carbonate; ammonium chloride; ammonium sulphate; ammonium hydroxide; and/or ammonium nitrate. Preferably, the ammonium compound is ammonium hydroxide

In an embodiment of the present invention the nitrogen-source may be supplied to the fermentation broth at a concentration below 0.1 g/l; e.g. below 0.09 g/l; such as below 0.08 g/l; e.g. below 0.07 g/l; such as below 0.06 g/l; e.g. below 0.05 g/l; such as 0.04 g/l; e.g. below 0.03 g/l; such as 0.02 g/l; e.g. below 0.01 g/l; such as 0.005 g/l; e.g. below 0.001 g/l.

In a further embodiment of the present invention the nitrogen-source may be supplied to the fermentation broth at a concentration in the range of 0.001-0.1 g/l; such as in the range of 0.005-0.09 g/l; e.g. in the range of 0.01-0.08 g/l; such as in the range of 0.02-0.075 g/l; e.g. in the range of 0.04-0.07 g/l; such as in the range of 0.05-0.06 g/l

In yet an embodiment of the present invention, the nitrogen-source supplied to the fermentation reactor may not be nitrate.

The nitrate concentration in the fermentation broth may be dependent on the biomass concentration. Hence, in a preferred embodiment of the present invention, the nitrate concentration in the fermentation broth may be maintained below 0.01 g nitrate/g biomass; such as below 0.008 g nitrate/g biomass; e.g. below 0.006 g nitrate/g biomass; such as below 0.004 g nitrate/g biomass; e.g. below 0.002 g nitrate/g biomass; such as below 0.001 g nitrate/g biomass; e.g. below 0.0005 g nitrate/g biomass; such as 0 g nitrate/g biomass. This calculation of the concentration of nitrate is based on a fermentation broth comprising viable methanotrophic microorganisms.

The carbon-substrate may preferably be a gaseous carbon-substrate.

Preferably, the carbon-substrate may be selected from an alkane, preferably, the alkane is a C1 compound. Even more preferably, the carbon-substrate may be methane, methanol, natural gas, biogas, syngas or any combination hereof. Even more preferably, the carbon-substrate may be methane.

As mentioned above the carbon-source and/or the nitrogen-source (as well as other ingredients added to the fermentation broth) may be added as a gas, there is a need to have these gases dissolved into the fermentation broth, which may be an aqueous fermentation broth, to be available for the microorganisms and available for the development of the biomass.

Generally, there is a challenge in the industry with the mass transfer of substrates (like, carbon-source; and oxygen source) and there are continuing interest and effort in improving this mass transfer. One way of improving fermentation in a U-loop fermenter may be described in WO 2010/069313 and/or WO 2003/016460, which are hereby incorporated by reference.

Thus, in the present invention the term “dissolved, or partly dissolved, in the fermentation broth” relates to the challenges known in the art with transforming the gaseous substrates from the gas phase into the aqueous phase, which is usable for the at least one microorganism.

In a preferred embodiment of the present invention, the nitrate concentration determined may be a dissolved nitrate concentration.

In a further embodiment of the present invention the nitrate concentration of the fermentation broth may be determined by an in-line analysis; by an on-line analysis; or by an off-line or at-line analysis. Preferably the nitrate concentration of the fermentation broth may be determined by an in-line analysis or by an on-line analysis.

In an even further embodiment of the present invention, the nitrate concentration of the fermentation broth may be continuously determined by an in-line analysis or by an on-line analysis.

In the context of the present invention, the term “in-line analysis” relates to a sensor that may be placed in a process vessel or stream of flowing material to conduct the analysis of one or more selected components.

In the context of the present invention the term “on-line analysis” relates to a sensor which may be connected to a process and conduct automatic sampling. On-line analysers may also be called in-line analysers.

On-line analysers and in-line analyses allow for continuous process control.

In the context of the present invention the terms “off-line analysis” or “at-line analyses” may be used interchangeably and relates to a sensor characterized by manual sampling followed by discontinuous sample preparation, measurement, and evaluation. The material properties can change during the time between sampling and the availability of the results, so direct process control may not be possible.

In an embodiment of the present invention, an oxygen-substrate may be supplied to the fermentation reactor. Preferably, the oxygen-substrate may be allowed to be dissolved, or partly dissolved, in the fermentation broth.

In a further embodiment of the present invention one or more nutrients; one or more pH adjusting components and/or water may be supplied to the fermentation reactor. The one or more nutrients; one or more pH adjusting components and/or water may preferably be allowed to be dissolved, or partly dissolved, in the fermentation broth.

The fermentation may be a batch fermentation, a fed-batch fermentation or a continuous fermentation. Preferably, the fermentation process may be a continuous fermentation process.

The methanotrophic organisms may preferably be a methanotrophic bacteria, such as Methylococcus capsulatus (used interchangeably with M. capsulatus).

The methanotrophic bacteria may be provided in a co-fermentation together with one or more heterotrophic bacteria.

The following heterotrophic bacteria may be particularly useful to co-ferment with M. capsulatus; Ralstonia sp.; Bacillus brevis; Brevibacillus agri; Alcaligenes acidovorans; Aneurinibacillus danicus and Bacillus firmus. Suitable yeasts may be selected from species of Saccharomyces and/or Candida.

The preferred heterotrophic bacteria are chosen from Alcaligenes acidovorans (NCIMB 13287), Aneurinibacillus danicus (NCIMB 13288) and Bacillus firmus (NCIMB 13289) and combinations thereof.

In an embodiment of the present invention, the methanotrophic organism may be a genetically modified methanotrophic organism and/or the heterotrophic organism may be a genetically modified heterotrophic organism.

The fermentation reactor and/or the fermentation process according to the present invention may have special relevance for the production of single cell protein (SCP) by continuous culture fermentation processes, e.g. by Methylococcus capsulatus.

The preferred methanotrophic bacteria are species of the Methylococcus family, especially Methylococcus capsulatus, which utilize methane or methanol as a carbon source and ammonia, nitrate or molecular nitrogen as a nitrogen source for protein synthesis.

A preferred embodiment of the present invention relates to a fermentation reactor comprising a loop-part and a top tank, said loop-part comprising a downflow part, connected to an upflow part via a U-part, wherein the top tank comprises:
(i) a first outlet connecting the top tank to the downflow part of the loop-part and allowing a fermentation liquid present in the top tank to flow from the top tank into the loop-part;
(ii) a first inlet connecting the top tank to the upflow part of the loop-part, allowing fermentation liquid present in the loop-part to flow from the loop part into the top tank;
(iii) a vent tube for discharging effluent gasses from the top tank; and
(iv) a visual inspection means.
   wherein the fermentation reactor further comprises:
(v) at least one inlet for supplying a substrate comprising an ammonium compound; and
(vi) at least sensor for determining the concentration of nitrate in the fermentation broth;

The fermentation reactor may preferably comprise at least one supply pump configured and/or controlled to automatically regulate the nitrate concentration in the fermentation broth.

In the present context the term “regulate the nitrate concentration” relates to the action of either reducing the nitrate concentration in the fermentation broth or increasing the nitrate concentration in the fermentation broth. Preferably, the term “regulate the nitrate concentration” relates to the action of reducing the nitrate concentration.

In an embodiment of the present invention the nitrate concentration in the fermentation broth may be regulated by regulating the flow of nitrogen source to the fermenter; regulating the flow of carbon-source to the fermenter; regulating the flow of oxygen; regulating the flow of nutrients; or a combination hereof.

The U-part of the loop-reactor may be connecting the lower part of the downflow part to the lower part of the upflow part. Furthermore, the upper part of the upflow part may be connected to the first inlet connecting the top tank to the upper part of the upflow part. The first outlet may be connecting the top tank to the upper part of the downflow part

In the present context the term “fermentation reactor” relates to a reactor comprising a top tank connected to the upper ends of a downflow part and an upflow part. The downflow part and the upflow part are connected at the lower ends via a U-part.

In the present context the term “loop reactor” relates to a specific example of a fermentation reactor.

The loop part of the present invention relates to the downflow part, the upflow part, as well as the connecting part at the lower ends of the upflow part and the downflow part formed by a U-part. Hence, the “loop part” relates to the fermentation reactor, without the top tank.

In the present context, the term “U-part” relates to bend provided in the bottom part of the fermentation reactor or the loop reactor connecting the lower ends of the upflow part and the downflow part. Preferably, the upflow part and the downflow part is vertical or substantially vertical.

In the present context, the term “top tank” relates to a container located at the top of the fermentation reactor and responsible for removal of effluent gas from the fermentation liquid. Preferably, the top tank is during operation/fermentation only partly filled with fermentation liquid. In an embodiment of the present invention the term “partly filled with fermentation liquid” relates to a 90:10 ratio between fermentation liquid and gas; such as an 80:20 ratio; e.g. an 70:30 ratio; such as an 60:40 ratio; e.g. an 50:50; such as an 40:60 ratio; e.g. an 30:70 ratio; such as an 20:80 ratio; e.g. an 10:90 ratio.

In the context of the present invention, the “visual inspection means” relates to one or more means allowing the skilled person to obtain direct information on the foaming characteristics in the top tank.

In an embodiment of the present invention, the direct information may be real-time information on the foaming characteristics in the top tank.

In a further embodiment of the present invention, the foaming characteristics in the top tank may involve, foaming density, foaming height, and level of turbulence provided in the top tank.

The turbulence in the top tank may be provided in the fermentation liquid present in the top tank when the fermentation liquid is forced from the upflow part through the first inlet and into the top tank.

The foaming density may be an expression of the size of the bubbles in the foam. The larger the bubbles in the foam the smaller the foaming density, smaller kg foam/m³. The smaller the bubbles in the foam the larger the foaming density, larger kg foam/m³.

In an embodiment of the present invention, the visual inspection means may be placed with a horizontal or substantial horizontal inspection view.

In a further embodiment of the present invention, the visual inspection means may be placed on the side of the top tank allowing a combined view above the surface of a fermentation liquid and below the surface of the fermentation liquid.

Preferably, the visual inspection means may be placed at the end of the top tank.

Even more preferably, the visual inspection means may be placed at the end of the top tank providing a view from the first inlet (or the upflow part) towards the first outlet (or the downflow part).

In an embodiment of the present invention, the visual inspection means may be an inspection hole, the camera, or a combination of an inspection hole and a camera.

Preferably, the inspection hole may be a sight glass.

The camera may be an inline camera.

In an embodiment of the present invention, the top tank may be provided with a light source in order to improve the visual inspection inside the top tank. The light source may be provided as a window allowing surrounding light to enter the top tank and/or as an artificial light source incorporated into the top tank.

In a further embodiment of the present invention, the light source may be provided as an individual feature (e.g. as an individual artificial light source) or as an integrated feature (e.g. as an integrated artificial light source) in the sight glass.

In addition to the visual inspection means the top tank may be provided with at least one foam sensor inside the top tank.

In order to avoid excessive foam development, a defoaming agent may be added to the fermentation liquid. Thus, the top tank may be provided with a defoaming inlet.

In an embodiment of the present invention the fermentation reactor, preferably the loop-part comprises an ion sensor or analyser for determining the content of one or more ion species in a fermentation liquid, preferably, the one or more ion species is selected from phosphate, calcium, hydrogen, nitrate, nitrite and/or ammonium, preferably nitrate and/or nitrite.

In a further embodiment of the present invention, the loop reactor may be provided with a circulation pump.

Preferably, the circulation pump may be placed in the upper half part of the downflow part.

In an embodiment of the present invention, the fermentation reactor may comprise a flow reducing device. Preferably, the flow reducing device may be inserted upstream from the first inlet and in the upper half of the upflow part.

In a further embodiment of the present invention, the loop-part of the fermentation reactor may preferably comprise one or more gas inlet; one or more water inlet; and/or one or more fermentation medium inlet.

The one or more gas inlet; the one or more water inlet; and/or the one or more fermentation medium inlet may be controlled by a computer. Preferably, the one or more gas inlet; the one or more water inlet; and/or the one or more fermentation medium inlet may be controlled by a computer based on the data obtained from the one or more sensors or analysers.

In order to provide improved fermentation conditions distribution of gaseous substrates, such as methane in the fermentation liquid may be important. Thus, the loop-part of the fermentation reactor may comprise one or more active devices for distributing gas in the fermentation liquid

In an embodiment of the present invention the one or more active devices for distributing gas in the fermentation liquid is a micro- or nano-sparger for introducing and/or distributing gas into the fermentation liquid; and/or a dynamic motion device placed in the loop part of the reactor, such as a dynamic mixer.

In addition to, or as an alternative to, the dynamic mixers, the loop-part may comprise one or more inactive mixing members. In an embodiment of the present invention, the one or more inactive mixing members may be a static mixer.

In addition to the importance of proper degassing in the top tank, it may be important to improve the mass transfer of the gaseous substrates into the liquid phase where the gas becomes available to the biocatalysts (e.g. the methanotrophic organisms) in an energy efficient manner.

Furthermore, as mentioned it may also be important to improve the efficiency of the waste gas removal by improving waste gas transfer from the liquid phase into the gas phase for removal from the fermenter, preferably done in the top tank.

Preferably, this improved efficiency in waste gas removal may be provided by operating the U-part of the loop part under increased pressure.

This improved mass transfer in combination with improved gas removal in the top tank may be achieved with the fermentation reactor, the loop reactor, according to the invention, which comprises a loop-part having an essentially vertical down-flow part, an essentially vertical up-flow part and a U-part having a substantially horizontal connecting part, which connects the lower end of the down-flow part with the lower end of the up-flow part, a top tank which may be provided above the loop-part and connects the upper end of the down-flow part and the upper end of the up-flow part.

In an embodiment of the present invention, the top tank may have a diameter which is substantially larger than the diameter of loop-part, the down-flow part, and/or the up-flow part.

In an embodiment of the present invention, the U-part of the fermenter may comprise an outlet, preferably placed in the top tank or in the U-part of the loop part of the fermentation reactor, for withdrawing fermentation liquid.

The fermentation reactor may comprise one or more gas injection points, which, according to wishes and demands, are placed in the down-flow part, the U-part and/or the up-flow part. Preferably, one or more gas injection points are placed in the down-flow part.

Directly following the one or more gas injection points, at least one active mixing members and/or at least one inactive mixing members for dispersion of the gas (or gasses) introduced into the fermentation liquid.

By increasing the pressure in the U-loop, loop reactor, an increased mass transfer from the gaseous phase to the liquid phase may be improved. Thus, a first pressure controlling device may be inserted in the U-part of the fermenter for increasing the pressure in at least a first zone of the U-part in the fermenter in relation to the pressure in a second zone of the fermenter.

In a preferred embodiment of the present invention, the first pressure controlling device may be inserted in the upper end of the down-flow part, and a second pressure controlling device may be inserted in the U-part of the fermenter and downstream of the first pressure controlling device when seen in the flow direction of the fermentation liquid.

The first pressure controlling device may be a valve (e.g. commercially available valve types), a pump, e.g. a propeller pump, a lobe pump, or a turbine pump, or the pressure may be increased by the injection of pressurized air or another gas, e.g. an inert gas. The first pressure controlling device is preferably a propeller pump, which also creates liquid circulation in the fermenter.

The second and optionally a third pressure controlling device may be placed in the down-flow part, the up-flow part, or in the U-part, but preferably the second pressure controlling device is in the upper half part of the up-flow part. The third optional pressure controlling device is preferably placed in the upper half part of the up-flow part and upstream to the second pressure controlling device when seen in the flow direction of the fermentation liquid. The second and/or third pressure controlling devices are chosen among a group of devices comprising a valve (e.g. commercially available valve types), a static mixer, a hydrocyclone, a pump (e.g. a propeller pump, a lobe pump or a turbine pump), a pressure controlled valve, a plate with holes, nozzles or jets or a narrowing of the diameter or cross-section of the fermenter part in which it is placed.

In an embodiment of the present invention, an improved mass transfer of the gaseous substrate may be provided in the U-part of the fermentation reactor according to the present invention.

In a further embodiment of the present invention, the waste gas removal may be provided in the top tank of the fermentation reactor according to the present invention.

In an embodiment of the present invention, means are provided in order to permit flushing of the headspace to improve waste gas removal and reduce the risk of explosive gas mixtures being formed in the headspace of the fermenter.

This flushing may be achieved by placing gas flushing means in the top tank, such as devices for adding and/or removing gas in a headspace. The gas flushing means may preferably be placed above the liquid surface for creating a gas flow of flushing gas co-currently, con-currently or cross-currently to the liquid flow in the top part of the fermenter. The gas adding means may also be placed below the liquid surface in the top part. Alternatively, or additionally, waste gas removal may be increased by reducing the pressure in the headspace by applying suction or a vacuum, thus reducing the pressure in the headspace and/or by installing flow modifying means in the top part. The invention also permits the energy applied to increase the pressure to be recovered for reuse. This may be achieved by connecting the second, and optionally the third pressure controlling device to a brake or a generator for decreasing the pressure with the propeller pump. If a generator is connected to the second and/or third pressure controlling device, some of the energy applied to the system may be collected, thus reducing the overall energy consumption of the system.

In the present context, the term “flushing” is used in respect of a process performed in the top tank for removing or assisting removal of effluent gas from the head space of the top tank and/or from the fermentation liquid in the top tank.

The top tank provided according to the present invention may be designed to contain between 1% and 99% of the overall volume of the fermenter, but preferably between 10% and 60% of the overall fermenter volume, even more preferably between 40-50% of the overall fermentation volume. In an embodiment of the present invention, the volume of the top tank may be less than the volume of the U-part.

The top tank may be provided with liquid or gas flow modifying means in order to assist mixing in the fermentation reactor or to assist gas bubble release from the fermentation liquid. The gas or liquid flow modifying means may be dynamic mixers, baffles or static mixers.

The size, i.e. both the diameter and the height of the fermenter may vary according to the needs of total fermenter volume.

In an embodiment of the present invention, the fermentation reactor according to the present invention may be provided with driving gas inlet where a driving gas may be introduced to drive carbon dioxide in the liquid phase into a separable effluent gas phase.

The driving gas inlet may preferably be placed upstream from the top tank and/or upstream from the first inlet.

The driving gas, i.e. the gas used to displace carbon dioxide from the dissolved phase (usually nitrogen but optionally another inert non-flammable gas) may, for example, be introduced at one or more points from the beginning of the substantially vertical up-flow zone to the entry into the effluent gas removal zone, however particularly preferably it will be introduced at one or more points between the upper portion (e.g. the upper 20%, more preferably the upper 10%) of the vertical portion of the up-flow zone and the beginning of the flattest (i.e. most horizontal) portion of the out-flow zone.

In the context of the present invention, the term “driving gas” is used in respect of a process performed in loop part, preferably in the upper end of the upflow part, and is assisting removal of effluent gas from the fermentation liquid into the gaseous phase.

In an embodiment of the present invention, the fermentation reactor includes both an inlet in the top tank for introducing a flushing gas into the top tank and an inlet in the upper end of the upflow part of the loop part for introducing a driving gas for moving effluent gas from the fermentation liquid into the gaseous phase.

One advantage of the present invention may be that an improved utilization of the gaseous substances added to the fermentation reactor may be provided.

The productivity of the fermentation reactor and/or the fermentation process according to the present invention may be further optimized in that the circulating fermentation liquid experiences an alternating pressure during circulation in the fermenter and has an increased mass transfer and solubility of substrate gases into the liquid phase in the zone having an increased pressure. The productivity may also be improved by the release of gases, such as waste gases from the circulating fermentation liquid, which release is increased in the zones where the pressure is reduced.

In an embodiment of the present invention the increased pressure in the loop part of the fermentation reactor, in the first zone and/or between the first pressure controlling device and the second pressure controlling device may be provided by applying a pressure above 0.5 bar above atmospheric pressure; such as a pressure above 1 bar above atmospheric pressure; e.g. a pressure above 1.5 bar above atmospheric pressure; such as a pressure above 2 bar above atmospheric pressure; e.g. a pressure above 2.5 bar above atmospheric pressure; such as a pressure above 3 bar above atmospheric pressure; e.g. a pressure above 3.5 bar above atmospheric pressure; such as a pressure above 4 bar above atmospheric pressure; e.g. a pressure above 4.5 bar above atmospheric pressure; such as a pressure above 5 bar above atmospheric pressure; e.g. a pressure above 5.5 bar above atmospheric pressure such as a pressure above 6 bar above atmospheric pressure; e.g. a pressure above 7 bar above atmospheric pressure.

In another embodiment of the present invention the increased pressure in the loop part of the fermentation reactor, in the first zone and/or between the first pressure controlling device and the second pressure controlling device may be provided by applying a pressure in the range of 0.5-10 bar above atmospheric pressure; such as a pressure in the range of 1-9 bar above atmospheric pressure; e.g. a pressure above 1.5-8 bar above atmospheric pressure; such as a pressure in the range of 2-7 bar above atmospheric pressure; e.g. a pressure above 3-6 bar above atmospheric pressure; such as a pressure in the range of 4-5 bar above atmospheric pressure.

In an even further embodiment of the present invention the pressure in the top tank may be less than 0.5 bar above atmospheric pressure; such as 0.25 bar above atmospheric pressure; such as 0.1 bar above atmospheric pressure; such as about atmospheric pressure; e.g. below 0.75 bar below atmospheric pressure; such as 0.5 bar below atmospheric pressure; e.g. below 0.25 bar below atmospheric pressure; such as 0.1 bar below atmospheric pressure.

Further details of suitable modifications to the loop reactor and feature on how to run such loop reactor, and processing of resulting biomass may be as described in WO 2010/069313; WO 2000/70014; WO 2003/016460; WO 2018/158319; WO 2018/158322; WO 2018/115042 and WO 2017/080987 which are all incorporated by reference.

An example of downstream processing suitable for the biomass obtained in order to provide various fraction may be as described in WO 2018/115042.

The sensors may include biosensors, electrochemical sensors, e.g. ion sensitive electrodes or sensors based on FIA (flow injection analysis) and optical measurements, e.g. spectrophotometric devices. A Near Infrared (NIR) probe may also be used for measuring several different components in the broth or in the cells in the fermenter, e.g. concentration of cells, amino acids, methanol, ethanol and/or different ions. The fermentation reactor may also be equipped with a mass spectrometric (MS) sensor or an electronic nose for determining the concentration of gaseous and volatile components (e.g. CO₂ and/or CH₄) in the headspace. The MS sensor or the electronic nose may control the pressure applied in the fermenter and/or the addition of gaseous components, e.g. methane and/or air/oxygen and/or the addition of gaseous ammonia or the ammonia/ammonium in solution. A high-speed camera may be installed in the U-part of the fermentation reactor, preferably in connection with gas injection, for determining the bubble size of the gases in the broth. The bubble size may be determined by image processing of the data from the high-speed camera.

The fermentation reactor according to the present invention may normally be run in continuous operation mode, after cleaning and a sterilization procedure, followed by a start period in which water, necessary nutrient salts, and the microorganisms are added to the fermentation reactor. The fermentation liquid may be circulated in the fermentation reactor, mainly by the first pressure controlling device. Then the addition of gaseous substrates may be initiated, and fermentation may be started. When the density of microorganisms has reached a concentration of approximately 0.5-10%, and preferably 1-5% (by dry weight) fermentation liquid may continuously be withdrawn from the fermentation reactor, e.g. from the top tank or from the U-part, and subjected to downstream processing, e.g. as described in WO 2018/115042

Withdrawing of fermentation liquid may be initiated simultaneously with the addition of make-up water, aqueous substrate and/or recirculation of supernatant at a dilution rate depending on the microorganisms used in the fermentation. Addition of substrate components in a liquid solution, additional water, recirculation of supernatant as make-up for the withdrawn broth and substrate gases may be controlled by a computer receiving data from the gas sensors and suitable calculations for providing the necessary amounts of each component for obtaining optimized growth of the organisms.

In an embodiment of the present invention, the fermentation process and the fermentation reactor may be a laboratory scale, a pilot plant and/or a production plant or an industrial plant. Preferably, the fermentation process and the fermentation reactor may be a production plant or an industrial plant

It should be noted that embodiments and features described in the context of one of the aspects of the present invention also apply to the other aspects of the invention.

All patent and non-patent references cited in the present application are hereby incorporated by reference in their entirety.

The invention will now be described in further details in the following non-limiting examples.

### EXAMPLES

### Example 1

The present example demonstrates the correlation between nitrate concentration in the fermentation broth and biomass development.

Nitrate formation was determined during the cultivation of M. capsulatus in 1 L BIOSAT® B-plus bioreactors (Sartorius, DK) where temperature was maintained at 42° C., agitation at 10 RPS⁻¹ (rounds per second) and pH at 6.7±0.05 by internal control loops adjusting cooling jacket water flow, motor frequency and dosing of 2M H₂SO₄ or 2M NaOH. Dissolved oxygen (DO) was monitored using a VisiFerm DO ECS 120 H₂ optical DO electrode (Hamilton, USA).

The bioreactors were continuously sparged with 96.81 g·h⁻¹ sterile air and 4.95 g·h⁻¹ of sterile methane (Instrument methane 3.5, AGA, DK).

The cultivation of M. capsulatus was initiated as a batch phase in 2NMS medium (Nitrate Mineral Salts medium) and continued under steady state (continuous phase fermentation) on AMS medium (Ammonium Mineral Salts medium) once nitrate was depleted. The feed flow rate during continuous cultivations was 48.95·10⁻³ Lh⁻¹. Cultures were brought to steady state before any attempt to induce co-metabolism were initiated.

Different pulse experiments of ammonia in the steady state has been carried out in 1 L fermenters under a fix condition where the biomass before and after the effect of the pulse together with nitrate concentration have been determined.

### Results

Tables 1 and 2 below shows that nitrate formation is increased with increasing ammonia concentration as a consequence of the pulse injection. The same experiment is maintained for 24 h where at higher concentration of ammonia pulse, biomass decreased suddenly, and it is almost near to wash out phase while nitrate was still there inside the reactor.

Tables 1 and 2: Different ammonia concentration fed in 1 L reactor under steady state and measure ammonia, nitrate and biomass concentration before injection of the ammonia injection, and at two different time points (at 2 hours after the pulse (table 1) and 24 hours after the pulse (table 2)).

TABLE 1
After 2 h

| Pulse of ammonia | Biomass-before | Biomass-after | Nitrate |
|---|---|---|---|
| (g/L) | (g/L) | (g/L) | (g/L) |
| 0.01 | 4.6 | 4.6 | 0 |
| 0.03 | 4.6 | 4.6 | 0.01 |
| 0.1 | 4.6 | 4.25 | 0.035 |

TABLE 2
After 24 h

| Pulse of ammonia | Biomass-before | Biomass-after | Nitrate |
|---|---|---|---|
| (g/L) | (g/L) | (g/L) | (g/L) |
| 0.01 | 4.6 | 4.6 | 0 |
| 0.03 | 4.6 | 4.6 | 0 |
| 0.1 | 4.6 | 2.02 | 0.029 |

Regulation of this high concentration of the nitrogen-source in the fermentation broth can be solved by regulating substrate flow rate to control the process such that no nitrate form and similarly no nitrite and/or nitrate accumulates. During these regulated conditions, any excess nitrate may be consumed by the M. capsulatus and the nitrogen concentration of the fermentation broth may be reduced.

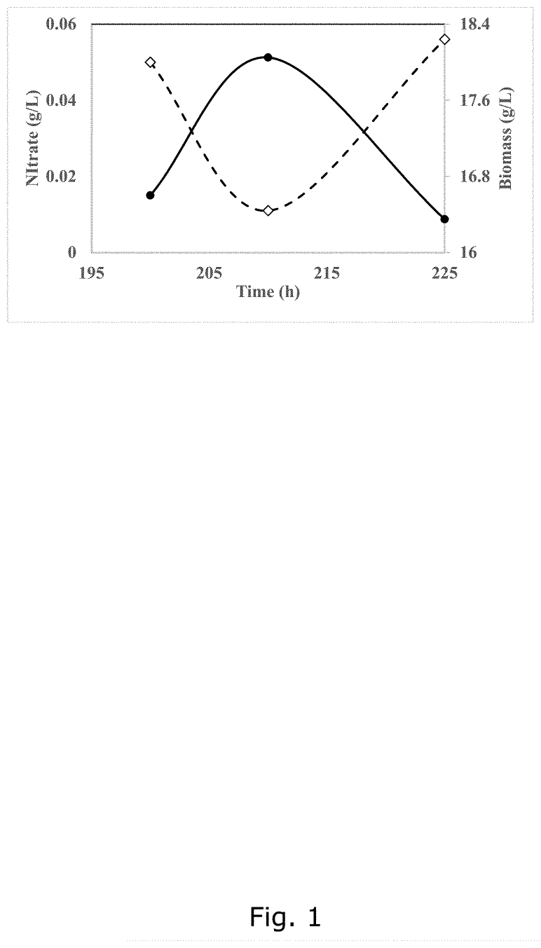

The same trend (excessive nitrate production leads to a decrease in biomass) have been observed in pilot plant as shown in FIG. 1. FIG. 1 shows that biomass production is going down as increasing nitrate production in the pilot plant and vice versa. The similar trend has been seen in the production plant and also in the lab as discussed in the Table 1 and 2.

### REFERENCES

WO 2010/069313
WO 2000/70014
WO 2003/016460
WO 2018/158319
WO 2018/158322
WO 2018/115042
WO 2017/080987
WO 2018/115042

1. A fermentation process for fermenting a fermentation broth comprising at least one microorganism in a fermentation reactor, wherein the fermentation process comprises the steps of:
a) supplying a carbon-substrate to the fermentation reactor allowing the carbon-substrate to be dissolved, or partly dissolved, in the fermentation broth;
b) supplying a nitrogen-substrate to the fermentation reactor allowing the nitrogen-substrate to be dissolved, or partly dissolved, in the fermentation broth; and
c) maintaining a nitrate concentration of the fermentation broth below 0.035 g/l, and/or maintaining a nitrate concentration of the fermentation broth below 0.01 g nitrate/g biomass;
wherein the at least one microorganism comprises at least one methanotrophic microorganism.

2. The fermentation process according to claim 1, wherein the nitrate concentration of the fermentation broth during fermentation is in the range of 0-0.035 g/l; e.g. in the range of 0.001-0.033 g/l; such as in the range of 0.002-0.03 g/l; e.g. in the range of 0.003-0.025 g/l; such as in the range of 0.004-0.02 g/l; e.g. in the range of 0.005-0.015 g/l; such as in the range of 0.007-0.01 g/l.

3. The fermentation process according to anyone of the preceding claims, wherein the nitrogen-source is selected from ammonia; ammonium compounds; and/or molecular nitrogen. Preferably, the nitrogen-source is ammonia.

4. The fermentation process according to anyone of the preceding claims, wherein the fermentation is a batch fermentation, a fed-batch fermentation or a continuous fermentation. Preferably, the fermentation process is a continuous fermentation process.

5. A fermentation reactor comprising a loop-part and a top tank, said loop-part comprising a downflow part, connected to an upflow part via a U-part, wherein the top tank comprises:
(i) a first outlet connecting the top tank to the downflow part of the loop-part and allowing a fermentation liquid present in the top tank to flow from the top tank into the loop-part;
(ii) a first inlet connecting the top tank to the upflow part of the loop-part, allowing fermentation liquid present in the loop-part to flow from the loop part into the top tank;
(iii) a vent tube for discharging effluent gasses from the top tank; and
(iv) a visual inspection means.
wherein the fermentation reactor further comprises:
(v) at least one inlet for supplying a substrate comprising an ammonium compound; and
(vi) at least sensor for determining the concentration of nitrate in the fermentation broth;

6. The fermentation reactor according to claim 5, wherein the fermentation reactor comprises at least one supply pump configured and/or controlled to automatically regulate the nitrate concentration in the fermentation broth.

7. The fermentation reactor according to anyone of claims 5-6, wherein the fermentation reactor is for the fermentation of methanotrophic organisms.

8. The fermentation reactor according to anyone of claims 5-7, wherein the fermentation reactor comprises an ion sensor or analyser for determining the content of one or more ion species in a fermentation liquid, preferably, the one or more ion species is selected from phosphate, calcium, hydrogen, nitrite and/or ammonium.

9. The fermentation reactor according to anyone of claims 5-8, wherein the loop-part of the fermentation reactor comprises one or more gas inlet; one or more water inlet; and/or one or more fermentation medium inlet.

10. The fermentation reactor according to claim 9, wherein the one or more gas inlet; the one or more water inlet; and/or the one or more fermentation medium inlet is controlled by a computer based on the data obtained from the one or more sensors or analysers.
